(19) United States
(12) Patent Application Publication
(10) Pub. No.: US 2026/0097194 A1
(43) Pub. Date: Apr. 9, 2026

(54) AUTOMATED DRUG MICROINJECTION DEVICE AND A METHOD OF OPERATING THE SAME

(71) Applicant: Pocket Clinic Corp., Toronto (CA)

(72) Inventors: Amir Hossein Omidvar, North York (CA); Atena Amanati Shahri, North York (CA)

(21) Appl. No.: 19/352,561

(22) Filed: Oct. 8, 2025

Related U.S. Application Data
(60) Provisional application No. 63/704,599, filed on Oct. 8, 2024.

(51) Int. Cl. A61M 37/00; A61M 5/142; A61M 5/158

(52) U.S. Cl. CPC A61M 37/0015; A61M 5/14212; A61M 5/158; A61M 2005/1585; A61M 2037/0023; A61M 2037/0061; A61M 2205/0266

(57) ABSTRACT

An automated drug microinjection device for administering a controlled amount of drug, and a method of operating the device is provided. The device comprises a container storing the drug, a microneedle assembly, a pump, a motor, and a controller. The microneedle assembly comprises a spring-loaded microneedle and a shape memory locking member. The spring-loaded microneedle is biased to be in an extended position and the shape memory locking member is configured to retain the microneedle in a locked and retracted position until a release signal is applied. The pump comprises a pump cavity and a pump actuator. The pump actuator is operable to fill the pump cavity with drug from the container and dispense the controlled amount of the drug from the pump cavity through the microneedle. The motor is configured to drive the pump actuator through a gear assembly comprising one or more gears integrated into the pump actuator.

### CROSS-REFERENCE TO RELATED APPLICATIONS

This application claims priority from U.S. provisional application No. 63/704,599, filed Oct. 8, 2024, the contents of which are hereby incorporated herein by reference.

### FIELD

The described embodiments relate to an automated drug microinjection device and a method of operating the same.

### BACKGROUND

Many health or medical-related conditions may require the administration of a drug/medication to a person for a long period of time (e.g., months/years/permanently). Globally, approximately one in three adults suffer from multiple chronic conditions (MCCs). As one non-limiting example, an estimated 537 million adults aged 20-79 are currently living with diabetes and this number is expected to rise to more than 1.3 billion by 2050. It is estimated that 79.4% of global diabetics live in low-and middle-income countries, with the life of more than 10% of these people depending on insulin administration to manage their condition. As other examples, the health/medical condition may include pulmonary arterial hypertension (PAH), Parkinson's disease, Alzheimer's disease, chronic pain condition, etc. The administered drug/medication may include a continuous or periodic (e.g., daily) injection to treat the above-noted or other health/medical conditions.

There can be many challenges associated with long-term administration of drugs/medication. For example, many insulin-dependent diabetics may rely on multiple daily injections (MDI) using syringes and pens that can cause underdose or overdose issues. Additional challenges can include long and arduous training periods, unpleasant psychological impact, and difficulties in conveyance, leading to a lack of treatment persistence and nonadherence, thereby creating barriers to achieving glycemic control. Insulin pumps may provide more accurate dosing and flexibility compared with MDI. However, the insulin pumps can have complex and non-user-friendly designs. Infusion sets can detach, leak, or cause skin irritation, thereby reducing the convenience associated with insulin pumps. Patch pumps aim to address some of these problems, but the initial acquisition and annual costs associated with the patch pumps can be significantly higher compared with MDI and thereby significantly increase the cost associated with the treatment.

### SUMMARY

The following summary is provided to introduce the reader to the more detailed discussion to follow. The summary is not intended to limit or define any claimed or as yet unclaimed invention. One or more inventions may reside in any combination or sub-combination of the elements or process steps disclosed in any part of this document including its claims and figures.

In a first aspect, there is provided an automated drug microinjection device for administering a controlled amount of drug. The device comprises a container storing the drug, a microneedle assembly, a pump, a motor, and a controller. The microneedle assembly comprises a spring-loaded microneedle and a shape memory locking member. The spring-loaded microneedle is biased to be in an extended position and the shape memory locking member is configured to retain the microneedle in a locked and retracted position until a release signal is applied. The pump comprises a pump cavity and a pump actuator. The pump actuator is operable to fill the pump cavity with drug from the container and dispense the controlled amount of the drug from the pump cavity through the microneedle. The motor is configured to drive the pump actuator through a gear assembly comprising one or more gears integrated into the pump actuator. The controller is configured to launch the microneedle into the extended position by applying the release signal to the shape memory locking member, provide a first motor control signal to operate the pump actuator to fill the pump cavity, and provide a second motor control signal to operate the pump actuator to dispense the controlled amount of the drug through the microneedle.

In a second aspect, there is provided a method of operating the automated drug microinjection device for administering a controlled amount of drug. The method comprises providing, by a controller of the device, a first motor signal to a motor to operate a pump actuator to fill a pump cavity of the device with the drug, the motor being configured to drive the pump actuator through a gear assembly comprising one or more gears integrated into the pump actuator; applying, by the controller, a microcontroller release a signal to a shape memory locking member to launch a spring-loaded microneedle from a retracted position to an extended position; providing, by the controller, a second motor signal to the motor to operate the pump actuator to dispense the controlled amount of the drug from the pump cavity through the microneedle.

In a third aspect, there is provided a method of use of an automated drug microinjection device for administering a controlled amount of a drug to a person. The method comprises: receiving a pump cartridge filled with the drug; installing the filled pump cartridge into the automated microinjection device; establishing a communication link between a user device and the automated microinjection device; positioning the automated microinjection device at a target user location; providing a microneedle actuation input from the user device to launch a microneedle of the automated microinjection device into the person; and providing a drug dispense input from the user device to a pump of the automated microinjection device to administer the controlled amount of the drug from the pump cartridge into the person via the pump and the microneedle.

### BRIEF DESCRIPTION OF THE DRAWINGS

The drawings included herewith are for illustrating various examples of systems, methods, and devices of the teaching of the present specification and are not intended to limit the scope of what is taught in any way.

FIG. 1 is a perspective view of an automated drug microinjection device, in accordance with an embodiment.

FIG. 2 is another perspective view of the device of FIG. 1, with a cover and upper housing components removed to show internal components of the device.

FIG. 3 is a cross-sectional perspective view of the device of FIG. 1.

FIG. 4 is a perspective view of an automated drug microinjection device, in accordance with another embodiment.

FIG. 5A is a cross-sectional view of a microneedle assembly of the device of FIG. 1 with a microneedle and a cannula in a locked and retracted position.

FIG. 5B is a cross-sectional view of the microneedle assembly of FIG. 5A with the microneedle and the cannula in an extended position.

FIG. 5C is a cross-sectional view of the microneedle assembly of FIGS. 5A and 5B with the microneedle in a partially retracted position and the cannula locked in the extended position.

FIG. 6 is a flowchart showing a method of use of the device of FIG. 1, in accordance with an embodiment.

FIG. 7 is a flowchart showing a method of controlling operations of the device of FIG. 1, in accordance with an embodiment.

### DETAILED DESCRIPTION

Several example embodiments are described herein. It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising” and variations thereof mean “including but not limited to,” unless expressly specified otherwise. A listing of items does not imply that any or all of the items are mutually exclusive, unless expressly specified otherwise. The terms “a,” “an” and “the” mean “one or more,” unless expressly specified otherwise.

As used herein and in the claims, two or more parts are said to be “coupled”, “connected”, “attached”, “joined”, “affixed”, “fastened”, or “mounted” where the parts are joined or operate together either directly or indirectly (i.e., through one or more intermediate parts), so long as a link occurs. As used herein and in the claims, two or more parts are said to be “directly coupled”, “directly connected”, “directly attached”, “directly joined”, “directly affixed”, “directly fastened”, or “directly mounted” where the parts are connected in physical contact with each other. As used herein, two or more parts are said to be “rigidly coupled”, “rigidly connected”, “rigidly attached”, “rigidly joined”, “rigidly affixed”, “rigidly fastened”, or “rigidly mounted” where the parts are coupled so as to move as one while maintaining a constant orientation relative to each other. None of the terms “coupled”, “connected”, “attached”, “joined”, “affixed”, “fastened” and “mounted” distinguish the manner in which two or more parts are joined together.

Further, although method steps may be described (in the disclosure and/or in the claims) in a sequential order, such methods may be configured to work in alternate orders. In other words, any sequence or order of steps that may be described does not necessarily indicate a requirement that the steps be performed in that order. The steps of methods described herein may be performed in any order that is practical. Further, some steps may be performed simultaneously.

As used herein and in the claims, a group of elements are said to ‘collectively’ perform an act where that act is performed by any one of the elements in the group, or performed cooperatively by two or more (or all) elements in the group.

Some elements herein may be identified by a part number, which is composed of a base number followed by an alphabetical or subscript-numerical suffix (e.g., 112a, or 112₁). Multiple elements herein may be identified by part numbers that share a base number in common and that differ by their suffixes (e.g., 112₁, 112₂, and 112₃). All elements with a common base number may be referred to collectively or generically using the base number without a suffix (e.g., 112).

As used herein and in the claims, “up”, “down”, “above”, “below”, “upwardly”, “vertical”, “elevation”, “upper”, “lower” and similar terms are in reference to a directionality generally aligned with (e.g., parallel to) gravity. The terms “distal”, “proximal” and similar terms are in reference to a directionality generally that is transverse (e.g., perpendicular) to gravity. However, none of the terms referred to in this paragraph imply any particular alignment between elements. For example, a first element may be said to be “vertically above” a second element, where the first element is at a higher elevation than the second element, and irrespective of whether the first element is vertically aligned with the second element.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural or object-oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Various embodiments have been described herein by way of example only. Various modifications and variations may be made to these example embodiments without departing from the spirit and scope of the invention, which is limited only by the appended claims.

The terms drug and medication are used interchangeably in this disclosure and may include any suitable fluid that is administered to a person. In some examples, the drug may be administered for treatment of a disease or medical condition. In some examples, the drug may be administered for management of a health condition (e.g., to address chronic pain). The terms drug and medication may include, but are not limited to, specific drugs or medication approved by a relevant regulatory authority (e.g., insulin, pain medication etc.).

The disclosed embodiments can provide a compact, user-friendly, and low-cost device for administration of a drug to a person. The disclosed device can administer a precise and controllable amount of drug. For example, other devices may be limited to administration of a fixed amount of a drug based on device design. In contrast, the disclosed device can administer a controllable amount of drug. For example, the disclosed device may receive a drug dose input specifying an amount of drug to be administered. The disclosed device can administer the precise amount of drug based on the drug dose input.

The disclosed device may include a disposable portion and a non-disposable portion. The disposable portion and the non-disposable portions can be detachably coupled to form a liquid-tight connection. The non-disposable portion may include electrical components, electrical boards, an energy storage device (e.g., a battery), a pump, and corresponding sub-components. The disposable portion may include a pump cartridge, a drug container (e.g., made using medical-grade silicone materials), a microneedle, and a cannula.

Medical-grade tubing may be used to connect the container to the pump and the pump to the microneedle. For example, PCT Publication No. WO2021/226683 describes medical-grade stainless steel tubing connections. The entire contents of PCT Publication No. WO2021/226683 is incorporated by reference herein.

The drug container may have any suitable capacity. For example, the drug container may have a capacity from 1 ml to 5 ml. In other examples, the drug container may have a higher capacity (e.g., 5 ml to 50 ml). One or more dimensions of the drug container may be controlled to change the capacity. For example, the capacity may be changed by only changing the height of the container/device while keeping other dimensions constant. In other examples, multiple dimensions of the container/device may be changed.

The disclosed device enables flexible and versatile operation by enabling easy replacement of the disposable portion. For example, drug containers of different capacities may be usable with the same disposable portion of the disclosed device.

The disclosed embodiments can provide a compact wearable device for drug administration. The device may be wearable for a suitable period before requiring replenishment. For example, the device may be wearable for a 5-10 day period before requiring replenishment. In other examples, the wearable period may be smaller than 5 days or greater than 10 days based on one or more factors including the drug container capacity, amount/rate of drug being administered, etc.

The disclosed device may be capable of communication with external devices including, for example, a remote server, a user device, a cloud-based monitoring system, etc. For example, the user device may include a smartphone or a smartwatch. A user may use a mobile app to provide a drug dose input to the disclosed device specifying an amount of drug to be administered. End to end data encryption techniques can be used to provide secure communication between the disclosed device and external devices.

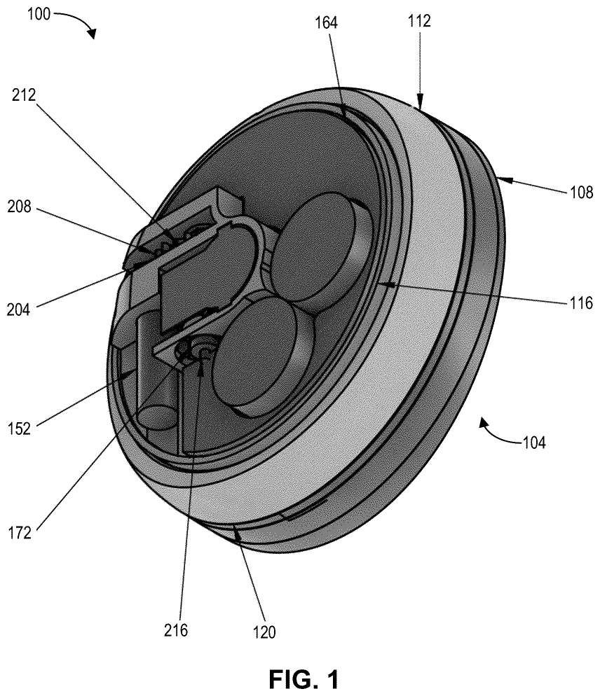

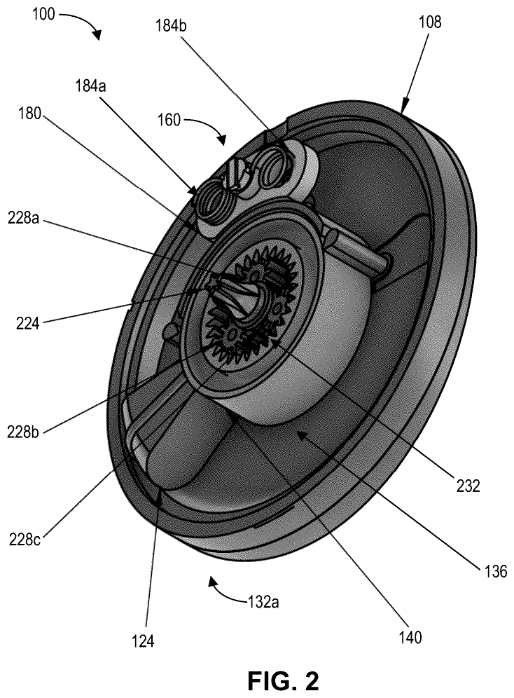

Reference is first made to FIGS. 1 and 2 showing an automated drug microinjection device 100, in accordance with an embodiment. FIG. 1 shows a perspective view of device 100. FIG. 2 shows another perspective view of device 100 with a cover and upper housing components removed to show internal components. In the illustrated embodiment, device 100 includes a housing 104, a pump 132, a motor 152, a gear assembly 156, a microneedle assembly 160, and a printed circuit board (PCB) 164.

Housing 104 may include any suitable design to house and/or protect the internal components of device 100. In some embodiments, housing 104 may be a disc-shaped housing having an oval, circular, or semi-circular perimeter to facilitate handling and use. In other embodiments, housing 104 may have a different shape and/or perimeter.

Housing 104 may include a lower housing 108, an upper housing 112, and a cover 116. Lower housing 108 may provide a casing for the disposable portion of device 100 (e.g., pump cartridge, drug container, microneedle assembly). Cover 116 and upper housing 112 may combine to provide a casing for the non-disposable portion of device 100 (e.g., pump, motor, gear assembly, PCB). Any suitable material may be used to manufacture housing 104 and its sub-components. For example, one or more of lower housing 108, upper housing 112, and cover 116 may be manufactured using medical-grade plastic materials.

Any suitable mechanism may be used to provide a liquid-tight sealing connection between upper housing 112 and lower housing 108. For example, upper housing 112 may include a metal ring 124 that abuts against a seal ring 128 of lower housing 108 to form a sealed connection when upper housing 112 and lower housing 108 are coupled together. Seal ring 128 may include, for example, a rubber gasket.

Any suitable mechanism may be used to couple upper housing 112 with lower housing 108 to provide the sealed connection. For example, a threaded connection may be used, and a twisting motion may push metal ring 124 against seal ring 128 to form a secured and sealed connection. This can enable a user-friendly design for installing a new pump cartridge into device 100 and for replacement/removal of a used pump cartridge. In other examples, other mechanisms may be used to generate the closing/sealing force between upper housing 112 and lower housing 108. For example, latches, clamps, cam locks, screws, magnets, adhesive, etc. may be used. In some embodiments, device 100 may include a detection mechanism to automatically detect whether the upper housing 112 and lower housing 108 are correctly coupled. An error signal may be generated in response to incorrect installation. An interlock mechanism may be implemented to prevent operation of device 100 until the error is rectified.

In some embodiments, device 100 may include an energy storage device. Any suitable energy storage device may be used, for example, a battery. The energy storage device may enable device 100 to be operable for a suitable time period before needing to be recharged. For example, the energy storage device may enable device 100 to operate for a period of 5 to 10 days before needing to be recharged. The energy storage device may be recharged using a wired or wireless connection.

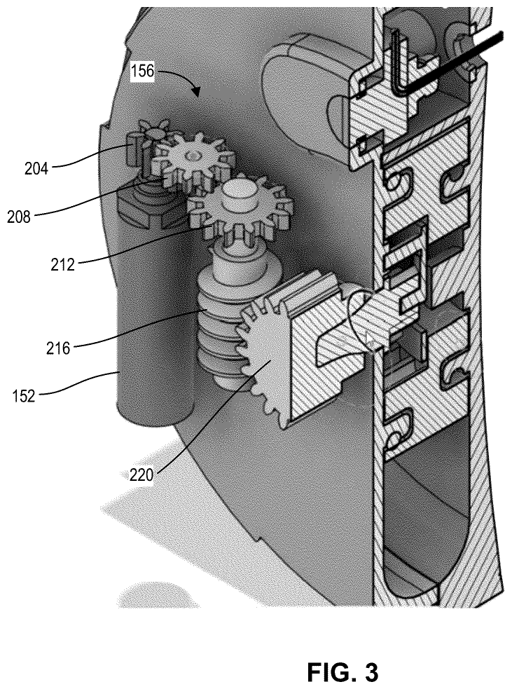

Any suitable pump design may be used. The pump may include a pump cavity and a pump actuator. The pump design may include for example, a rotary vane pump, a peristaltic pump or a piston pump. The rotary vane pump may include a rotary vane pump actuator. The peristaltic pump may include multiple rollers and flexible tubing. The piston pump may include a piston pump actuator and a pump cavity formed as a bladder. Reference is now additionally made to FIG. 3 showing a cross-sectional perspective view of device 100. FIGS. 1 to 3 show an embodiment where device 100 includes a rotary vane pump 132a.

Rotary vane pump 132a may include a pump cavity 136 and a rotary vane 140. Pump cavity 136 may be in fluid communication with the drug container through a first control valve and with the microneedle through a second control valve. Any suitable valve interlock mechanism (e.g., a mechanical or electrical locking mechanism) may be implemented so that the first control valve and the second control valve are not open at the same time. This can avoid direct uncontrolled flow between the drug container and the microneedle.

Device 100 may include any suitable motor 152 to drive the pump actuator. For example, device 100 may include a stepper motor 152. Stepper motor 152 may receive control inputs from PCB 164. In other examples, a different motor may be used, such as, for example, a Brushless DC motor, Ultrasonic motor, a Piezo Actuator, etc.

Gear assembly 156 may be used to transfer output of stepper motor 152 to rotary vane 140. Gear assembly 156 can enable precise control of rotary vane 140. Gear assembly 156 may include any suitable combination of gears. In the illustrated embodiment, gear assembly 156 includes a spur gear 204 having 6 teeth, a spur gear 208 having 10 teeth, a spur gear 212 having 13 teeth, a worm gear 216, and a spur gear 220 having 20 teeth.

Gear assembly 156 further includes a planetary gear assembly that is integrated into rotary vane 140. The planetary gear assembly includes a sun gear 224, multiple planet gears 228a-228c and ring gear 232. The integration of the planetary gear assembly into rotary vane 140 can enable a compact design of device 100 while providing sufficient torque and pressure to administer high viscosity fluids.

Any suitable gearing ratios may be used to enable administration of the drug with sufficient torque and pressure. For example, a gearing ratio of 1:5 may be used between the worm gear and the sun gear to provide at least 35 psi pressure for drug administration. In other examples, different gearing ratios may be used based on space constraints and torque and pressure requirements.

An initial rotation of rotary vane 140 can enable pump cavity 136 to be filled with drug from the drug container. Motor 152 and gear assembly 156 can enable precise rotation control of rotary vane 140 to administer precise and controlled amounts of the drug from pump cavity 136 through the microneedle.

In other embodiments, gear assembly 156 may include any other number and/or type of gears. For example, the planetary gear assembly may include a greater number of planet gears. As another example, spur gears 204-212 may have different number of teeth.

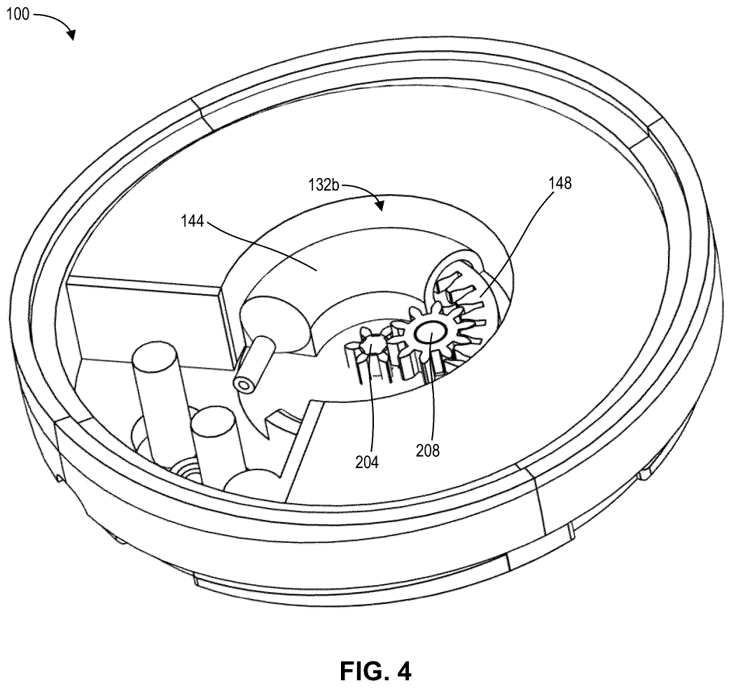

Reference is now made to FIG. 4. FIG. 4 shows a perspective view of another embodiment of device 100 with housing components removed to show internal components. The device 100 shown in FIG. 4 may include a housing, a pump, a motor, a gear assembly, a microneedle assembly, and a PCB, as described herein above with reference to FIGS. 1 to 3. For the embodiment illustrated in FIG. 4, device 100 includes a piston pump 132b. Piston pump 132b may include a bladder 144 and a piston 148.

Similar to the pump cavity of the rotary vane pump, bladder 144 may be in fluid communication with the drug container through a first control valve and with the microneedle through a second control valve. Any suitable valve interlock mechanism (e.g., a mechanical or electrical locking mechanism) may be implemented so that the first control valve and the second control valve are not open at the same time. This can avoid direct uncontrolled flow between the drug container and the microneedle.

A gear assembly (e.g., including gears 204 and 208 shown in FIG. 4) may be used to transfer output of a motor to piston 148. The gear assembly can enable precise control of piston 148 and may be integrated into piston 148 as shown in FIG. 4. The integration of the gear assembly into piston 148 can enable a compact design of device 100 while providing sufficient torque and pressure to administer high viscosity fluids. The gear assembly can enable precise control of a volume of bladder 144. Piston 148 may initially be actuated to fill bladder 144 with the drug from the drug container. The motor and gear assembly can enable precise actuation of piston 148 to administer precise and controlled amounts of the drug from bladder 144 through the microneedle.

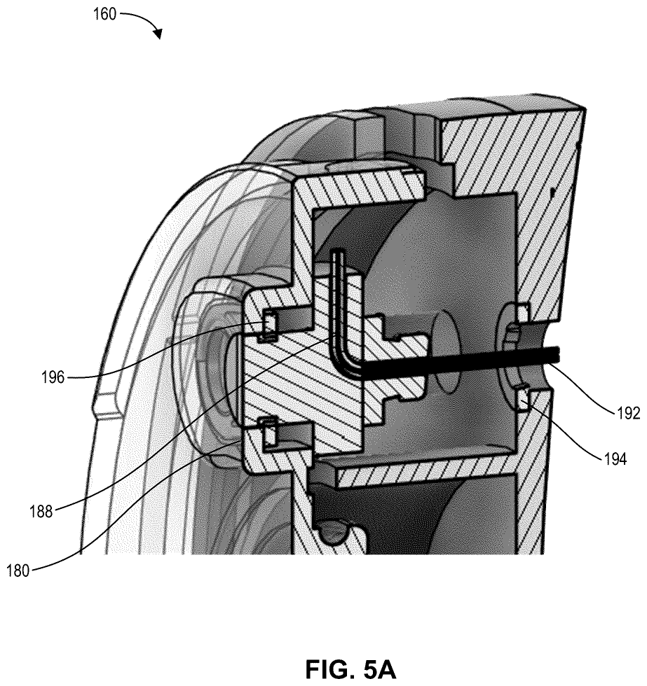

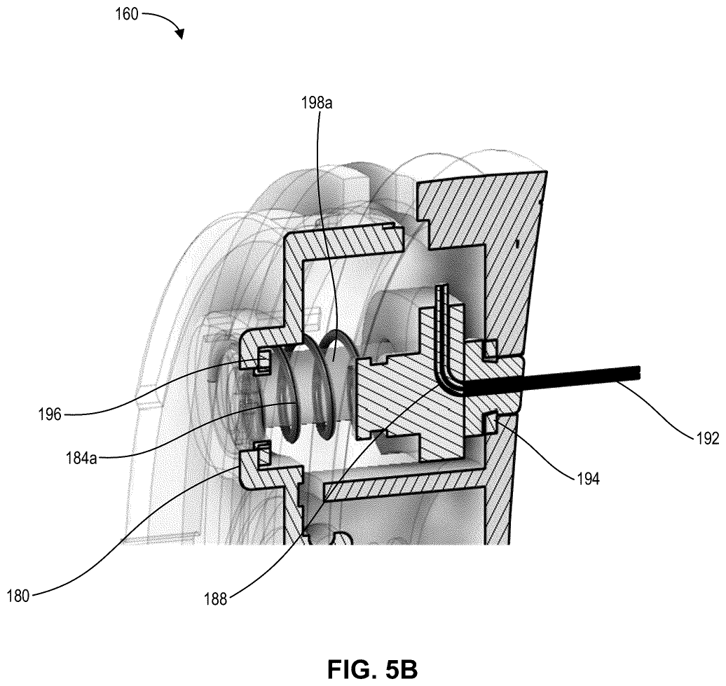

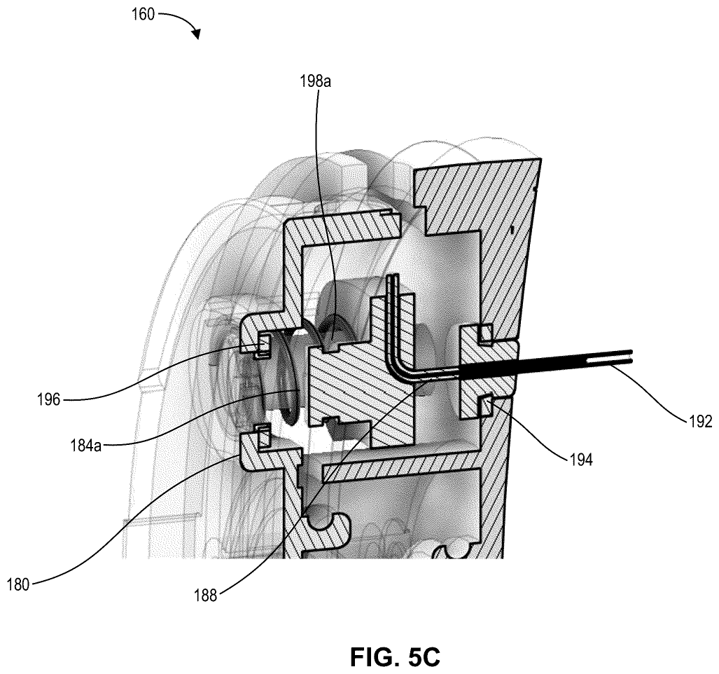

Reference is now made to FIGS. 5A-5C showing cross-sectional views of microneedle assembly 160. FIG. 5A shows the microneedle and cannula in a locked and retracted position. FIG. 5B shows the microneedle and cannula in an extended position. FIG. 5C shows the microneedle in a partially retracted position and the cannula locked in the extended position.

Microneedle assembly 160 may include a needle hub 180, a pair of springs 184 (e.g., spring 184a and spring 184b shown in FIG. 2), a pair of actuating rods 198 (e.g., one actuating rod 198a shown in FIG. 5B), a lock 196, a microneedle 188, a cannula 192, and a locking pin 194.

Needle hub 180 may provide compact integration of other components of microneedle assembly 160 into device 100. Microneedle 188 and cannula 192 may be in fluid communication with the pump cavity. Microneedle 188 may be made using any suitable material that provides sufficient mechanical strength and rigidity. For example, microneedle 188 may be made of medical grade stainless steel material. Cannula 192 may be made using any suitable flexible material, for example, a silicone material.

Lock 196 may have any suitable design to retain microneedle 188 and cannula 192 in a retracted position against the pressure of springs 184. For example, FIG. 5A shows lock 196 retaining microneedle 188 and cannula 192 in a locked and retracted position. In some embodiments, lock 196 may be made using a shape memory material.

Springs 184 may provide sufficient pressure via actuating rods 198 to inject microneedle 188 and cannula 192 into a person to administer the drug. For example, FIG. 5B shows microneedle 188 and cannula 192 in an extended position. Further, microneedle 188 may be retracted leaving cannula 192 inserted into the person. For example, FIG. 5C shows microneedle 188 in a partially retracted position corresponding to a free length of springs 184. Locking pin 194 can have any suitable design to retain cannula 192 in the extended position.

Microneedle assembly 160 may enable launching of microneedle 188 and cannula 192 with sufficient insertion force using a small control signal. This can provide a user-friendly design where a large user force is not required to launch needle insertion. For example, a control signal (e.g., a voltage or a current) may be applied to the shape memory material. The control signal can cause corresponding current and heat generation in the shape memory material, thereby causing the shape memory material to change shape. Lock 196 can be structurally designed to release the lock on microneedle 188 and cannula 192 when the shape memory material changes shape. Springs 184 can provide the required insertion force for microneedle 188 and cannula 192. In other embodiments, different structures may be used to implement the locking mechanism for microneedle 188 and cannula 192.

Reference is now made to FIGS. 1 to 5B. PCB 164 may include one or more electronic components. In some embodiments, PCB 164 may include a combination of multiple boards, each board design to provide specific functionalities. PCB 164 may include a communication component to provide bidirectional communication with one or more external devices. For example, PCB 164 may enable device 100 to receive a drug dose input.

PCB 164 may further include one or more processors. The processor may provide a control signal to motor 152 to actuate pump 132 to administer a drug amount specified by the drug dose input.

PCB 164 may receive a sensor input indicating a position of rotary vane 140/piston 148 of pump 132. For example, PCB 164 may receive an input from a rotary encoder indicating the position of rotary vane 140 of rotary vane pump 132a. PCB 164 may determine a final position for rotary vane 140 based on an initial position of rotary vane 140, an amount of drug to be administered and a transfer function defining the relationship between amount of drug administrated and degree of rotation of rotary vane 140. For example, the transfer function may define that 0.00152 ml of insulin is administered for 0.1 degree of rotation of rotary vane 140. In other examples, the transfer function may be different based on one or more factors including, for example, drug properties (e.g., viscosity), volume of the pump cavity, rotary vane size etc.

In some embodiments, PCB 164 may include one or more components to apply a control signal to microneedle assembly 160 to launch microneedle 188 and cannula 192. For example, a current driver may provide an input current signal to lock 196 of microneedle assembly 160 based on a processor output.

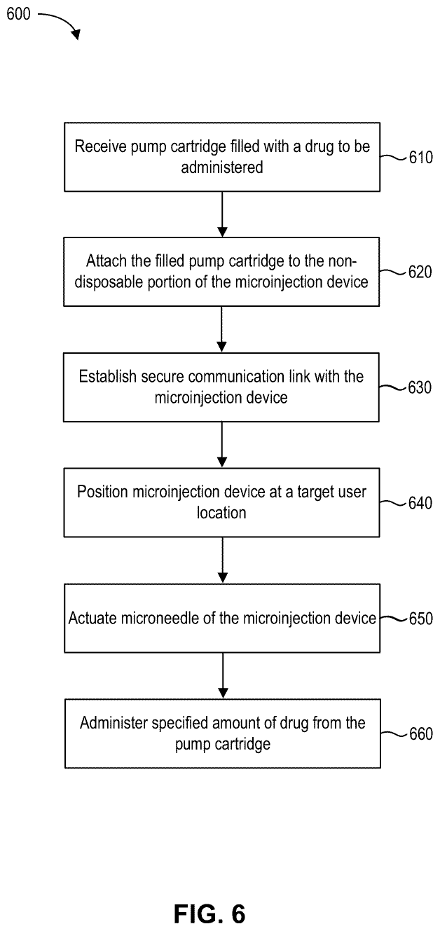

Reference is now made to FIG. 6. FIG. 6 is a flowchart showing a method 600 of use of an automated drug microinjection device. The automated drug microinjection device can be, for example, device 100 shown in FIGS. 1 to 5 and concurrent reference is made to device 100 and its components shown in FIGS. 1 to 5.

In some examples, method 600 may be executed by a user to self-administer a drug. In some examples, method 600 may be executed by a medical services provider to administer a drug to a person.

At act 610, a filled pump cartridge may be received. For example, the user may receive, from a medical services provider, a pump cartridge filled with a drug to be administered. As another example, the user may fill an empty pump cartridge with a drug to be administered.

At act 620, the filled pump cartridge may be attached to the non-disposable portion of device 100. For example, the user may install the filled pump cartridge into housing 104 and form a liquid-tight sealing connection between upper housing 112 and lower housing 108 as described herein.

At act 630, a secure communication link may be established with the device. For example, device 100 may be paired with a user device (e.g., a smartphone of the user). The user may provide one or more inputs to device 100 using the secure communication link. For example, the user may use an app on the paired smartphone to provide a dose input specifying an amount of the drug to be administered.

At act 640, the device may be positioned at a target user location. For example, the user may attach device 100 at a desired location on their body. Device 100 may be attached using any suitable mechanism, for example, using an adhesive.

At act 650, a microneedle of the device may be actuated to launch the microneedle and insert a cannula into the user. For example, the user may actuate the microneedle using a digital button provided via a user interface of the user device paired with device 100. As another example, the user may actuate the microneedle using a physical button or switch located on device 100 (e.g., located at housing 104).

At act 660, a specified amount of drug may be administered from the pump cartridge and through the microneedle and cannula to the user. The user may specify the drug amount to be administered using the paired user device. Act 660 may be performed multiple times until there is insufficient amount of drug remaining in the pump cartridge for further administration. A user may remove the used pump cartridge from device 100 and method 600 may proceed to act 610 to receive a filled pump cartridge.

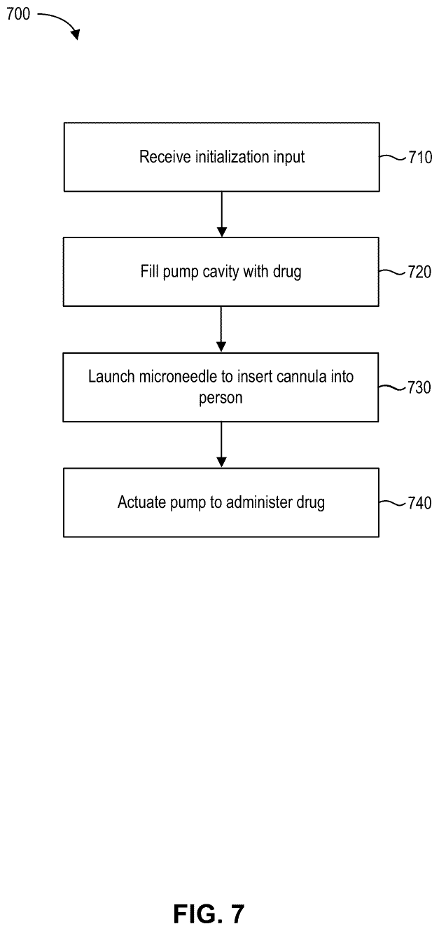

Reference is now made to FIG. 7. FIG. 7 is a flowchart showing a method 700 of controlling operations of an automated drug microinjection device. The automated drug microinjection device can be, for example, device 100 shown in FIGS. 1 to 5 and concurrent reference is made to device 100 and its components shown in FIGS. 1 to 5. Method 700 may be performed, for example, by a controller of device 100 (e.g., PCB 164) to administer a controlled amount of drug to a person using device 100 attached to the person.

At act 710, an initialization input may be received. For example, device 100 may receive inputs specifying one or more of the drug to be administered, a drug dose input specifying the amount of drug to be administered and a transfer function specifying the relationship between pump actuation and amount of drug that is administered. The inputs may be received, for example, from a user device (e.g., a smartphone) that is paired with device 100. The inputs may be received using any suitable wireless (e.g., Bluetooth™, IEEE 802.15.4 wireless standards such as ZigBee 3.0 and Open thread) or wired communication technology.

At act 720, the pump cavity may be filled with the drug to be administered. For example, device 100 may receive a fill input. In some embodiments, the fill input may be received from an external device (e.g., from a user device like a smartphone). In some embodiments, the fill input may be received from the activation of a switch or a button of device 100. In response to a received input, a controller of device 100 (e.g., PCB 164) may provide a motor control signal to motor 152 to actuate pump 132. For a rotary vane pump 132a, motor 152 may control movement of rotary vane 140 to fill pump cavity 136 with drug from the drug container of the drug cartridge. For a piston pump 132b, motor 152 may control movement of piston 148 to fill bladder 144 with drug from the drug container of the drug cartridge.

At act 730, the microneedle may be launched to insert the cannula into the person. Act 730 may be performed in response to a needle launch input. In some embodiments, the needle launch input may be an activation of a switch or a button of device 100. For example, the person may press a pushbutton located on housing 104 of device 100 to launch the microneedle and cannula. In some embodiments, the needle launch input may be received from an external device (e.g., from a user device like a smartphone or a smartwatch).

In response to a received needle launch input, a controller of device 100 (e.g., PCB 164) may provide a control signal (e.g., a control voltage or current) to lock 196 of microneedle assembly 160. The control signal can release lock 196 and springs 184 can provide the required insertion force for microneedle 188 and cannula 192.

At act 740, the pump may be actuated to administer the drug to the person. Act 740 may be performed in response to a drug dose input. The drug dose input may be received from an external device (e.g., from a user device like a smartphone or a cloud-based server). The drug dose input may specify an amount of drug to be administered. The drug dose input may be received using any suitable wireless or wired communication technology.

In response to a received drug dose input, a controller of device 100 (e.g., PCB 164) may determine the pump actuation required to administer the specified amount of drug. For example, the pump actuation may be determined as an amount of rotation of rotary vane 140 of rotary vane pump 132a or a displacement amount of piston 148 of piston pump 132b. Based on the determined amount of required pump actuation, the controller can provide a motor control signal to motor 152 to actuate pump 132 to administer the drug.

Method 700 may repeat acts 720 and 740 multiple times. For example, act 740 may be executed multiple times until there is insufficient amount of drug remaining in the pump cavity for a further administration. Method 700 may then proceed to act 720 to refill the pump cavity with the drug. Acts 720 and 740 may be executed multiple times until there is insufficient amount of drug stored in the drug container for future administration. The disposable portions of the device 100 including the drug cartridge, the microneedle and the cannula may be replaced when there is insufficient amount of stored drug remaining. After replacement of the disposable portions, method 700 may be further executed through acts 710-740 as described herein above.

The present invention has been described here by way of example only. Various modification and variations may be made to these exemplary embodiments without departing from the spirit and scope of the invention, which is limited only by the appended claims.

We claim:

1. An automated drug microinjection device for administering a controlled amount of drug, the device comprising:
a container storing the drug;
a microneedle assembly comprising a spring-loaded microneedle and a shape memory locking member, the spring-loaded microneedle being biased to be in an extended position and the shape memory locking member being configured to retain the microneedle in a locked and retracted position until a release signal is applied;
a pump comprising a pump cavity and a pump actuator, the pump actuator operable to fill the pump cavity with drug from the container and dispense the controlled amount of the drug from the pump cavity through the microneedle;
a motor configured to drive the pump actuator through a gear assembly comprising one or more gears integrated into the pump actuator; and
a controller configured to:
launch the microneedle into the extended position by applying the release signal to the shape memory locking member;
provide a first motor control signal to operate the pump actuator to fill the pump cavity; and
provide a second motor control signal to operate the pump actuator to dispense the controlled amount of the drug through the microneedle.

2. A method of operating an automated drug microinjection device for administering a controlled amount of drug, the method comprising:
providing, by a controller of the device, a first motor signal to a motor to operate a pump actuator to fill a pump cavity of the device with the drug, the motor being configured to drive the pump actuator through a gear assembly comprising one or more gears integrated into the pump actuator;
applying, by the controller, a microneedle release signal to a shape memory locking member to launch a spring-loaded microneedle from a retracted position to an extended position;
providing, by the controller, a second motor signal to the motor to operate the pump actuator to dispense the controlled amount of the drug from the pump cavity through the microneedle.

3. A method of use of an automated drug microinjection device for administering a controlled amount of a drug to a person, the method comprising:
receiving a pump cartridge filled with the drug;
installing the filled pump cartridge into the automated microinjection device;
establishing a communication link between a user device and the automated microinjection device;
positioning the automated microinjection device at a target user location;
providing a microneedle actuation input from the user device to launch a microneedle of the automated microinjection device into the person; and
providing a drug dispense input from the user device to a pump of the automated microinjection device to administer the controlled amount of the drug from the pump cartridge into the person via the pump and the microneedle.
